(19) United States
(12) Patent Application Publication
(10) Pub. No.: US 2007/0249953 A1
(43) Pub. Date: Oct. 25, 2007

(54) METHOD AND APPARATUS FOR DETECTION OF NERVOUS SYSTEM DISORDERS

(75) Inventors: Mark G. Frei, Lawrence, KS (US); Ivan Osorio, (US); David L. Carlson, Fridley, MN (US); Eric J. Panken, Edina, MN (US); Touby A. Drew, Minneapolis, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: MEDTRONIC, INC., Minneapolis, MN (US)

(21) Appl. No.: 11/609,388

(22) Filed: Dec. 12, 2006

Related U.S. Application Data
(60) Provisional application No. 60/793,998, filed on Apr. 21, 2006.

(51) Int. Cl. A61B 5/04; A61N 1/00

(52) U.S. Cl. 600/544; 604/19; 607/2

(57) ABSTRACT

Systems and methods for detecting and/or treating nervous system disorders, such as seizures, are disclosed. Certain embodiments of the invention relate generally to implantable medical devices (IMDs) adapted to detect and treat nervous system disorders in patients with an IMD. Certain embodiments of the invention include detection of seizures based upon comparisons of long-term and short-term representations of physiological signals. Other embodiments include prediction of seizure activity based upon analysis of physiological signal levels. A further embodiment of the invention includes detection of seizure activity following the delivery of therapy.

### CROSS REFERENCE TO RELATED APPLICATIONS

This application claims priority to U.S. Provisional Patent Application Ser. No. 60/793,998, filed on Apr. 21, 2006.

### FIELD OF THE INVENTION

The present invention relates generally to implantable medical devices (IMDs), and more particularly relates to systems and methods for detecting and/or treating nervous system disorders, such as seizures, in a patient with an IMD.

### BACKGROUND OF THE INVENTION

Nervous system disorders affect millions of people, causing a degradation of life, and in some cases, death. Nervous system disorders may include disorders of the central nervous system and the peripheral nervous system. Such disorders may include, for example without limitation, epilepsy, Parkinson's disease, essential tremor, dystonia, and multiple sclerosis (MS). Additionally, nervous system disorders may also include mental health disorders and psychiatric disorders, which also affect millions of individuals and include, but are not limited to, anxiety (such as general anxiety disorder, panic disorder, phobias, post traumatic stress disorder (PTSD), and obsessive compulsive disorder (OCD)), mood disorders (such as major depression, bipolar depression, and dysthymic disorder), sleep disorders (e.g., narcolepsy), obesity, and anorexia.

As an example, epilepsy is a serious nervous system disorder, which is prevalent across all ages. Epilepsy is a group of neurological conditions in which a person has or is predisposed to recurrent seizures. A seizure is a clinical manifestation resulting from excessive, hypersynchronous, abnormal electrical or neuronal activity in the brain. A seizure is a type of adverse neurological event that may be indicative of a nervous system disorder. This electrical excitability of the brain may be likened to an intermittent electrical overload that manifests with sudden, recurrent, and transient changes of mental function, sensations, perceptions, and/or involuntary body movement. Because the seizures are unpredictable, epilepsy affects a person's employability, psychosocial life, and ability to operate vehicles or power equipment. Epilepsy is a nervous system disorder that occurs in all age groups, socioeconomic classes, cultures, and countries. In developed countries, the age-adjusted incidence of recurrent unprovoked seizures ranges from 24/100,000 to 53/100,000 person-years and may be even higher in developing countries. In developed countries, age-specific incidence is highest during the first few months of life and again after age 70. The age-adjusted prevalence of epilepsy is 5 to 8 per 1,000 (0.5% to 0.8%) in countries where statistics are available. In the United States alone, epilepsy and seizures affect 2.3 million Americans, with approximately 181,000 new cases occurring each year. It is estimated that 10% of Americans will experience a seizure in their lifetimes, and 3% will develop epilepsy by age 75.

There are various approaches in treating nervous system disorders. Treatment therapies can include any number of possible modalities alone or in combination including, for example, electrical stimulation, magnetic stimulation, and/or drug infusion. Each of these treatment modalities can be operated using closed-loop feedback control. Such closed-loop feedback control techniques may receive signals (e.g., neurological signals from a monitoring element) carrying information about a symptom or a condition or a nervous system disorder. Such a neurological signal can include, for example, electrical signals (such as electroencephalogram (EEG), electrocorticogram (ECoG), and/or electrocardiogram (EKG) signals), chemical signals, other biological signals (such as changes in the quantity of neurotransmitters), temperature signals, pressure signals (such as blood pressure, intracranial pressure or cardiac pressure), respiration signals, heart rate signals, pH-level signals, and peripheral nerve signals (such as cuff electrodes placed on a peripheral nerve). Monitoring elements can include, for example, recording electrodes or various types of sensors.

For example, U.S. Pat. No. 5,995,868 to Dorfineister et al., incorporated herein by reference in relevant part, discloses a system for the prediction, rapid detection, warning, prevention, or control of changes in activity states in the brain of a patient. Use of such a closed-loop feedback system for treatment of a nervous system disorder may provide significant advantages. For example, it may be possible for treatment to be delivered before the onset of the symptoms of the nervous system disorder, potentially preventing such symptoms from occurring.

In the management of a nervous system disorder, it may be important to determine and/or assess the extent of a neurological event, the location of the neurological event, the severity of the neurological event, and the occurrence of multiple (possibly related) neurological events in order to prescribe and/or provide the delivery of a treatment, or otherwise manage the nervous system disorder. A patient, for example, would not benefit from a medical device system if the patient experienced a neurological event, but was not administered treatment because the medical device system did not detect the neurological event. On the other hand, a patient may suffer adverse effects, for example, if subjected to a degree of treatment corresponding to a severe neurological event, or to multiple neurological events, such as seizures, when in fact the patient had experienced only one neurological event, or a series of minor events, or no neurological event at all. As used herein, the term “neurological event” may encompass physiological events, such as seizures, as well as events defined artificially, for example, by measurable signal processing parameters.

Glossary of Terms

The “onset of the clinical component” of a seizure is the earlier of either (1) the time at which a patient becomes aware that a seizure is beginning (the “aura”), or (2) the time at which an observer recognizes a significant physical or behavioral change typical of a seizure.

The “onset of the electrographic component” of a seizure is defined by the appearance of a class of signal changes recognized as characteristic of a seizure. This analysis may typically include visual review of signal tracings of varying duration, both before and after the perceived signal changes, using multiple channels of information and clinical correlates. The precise determination of the onset is subject to personal interpretation, and may vary based on the skill and attention level of the reviewer, the quality of data, and the nature and format of the data displayed.

An electroencephalogram, or EEG, usually refers to voltage potentials recorded from the scalp. The term “EEG” typically encompasses recordings made outside the dura mater. The electrocorticogram, or ECoG, typically refers to voltage potentials recorded intracranially, e.g., directly from the cortex. It should be noted that the methods and devices described herein may be applied to any signal representing electrical activity sensed from a patient's brain, including EEG and ECoG signals. For simplicity, the term “EEG” has been used throughout this disclosure, and is intended to encompass EEG and ECoG types of signals, as well as any other signals representing electrical activity sensed from a patient's brain.

The period of time during which a seizure is occurring is called the ictal period. Those skilled in the art will appreciate that the term “ictal” may also be used to refer to phenomena other than seizures. Periods of time when a patient is not in a state of seizure, or in transition into or out of the seizure state, are known as “interictal” periods.

The term “false positive” refers to the case of a system mistakenly detecting a non-seizure signal and classifying it as a seizure. The term “false negative” describes the case in which a true seizure goes undetected by a system. Systems that have a low rate of false positive detections are called specific, while those with a low rate of false negative detections are called sensitive.

The term “epileptiform discharge” is used herein to refer to a class of sharply contoured waveforms, usually of relatively high signal energy, having a relatively brief duration (e.g., rarely exceeding about 200 msec). These epileptiform discharge signals (or “spikes”) can form complexes with slow waves, and can occur in singlets or in multiplets.

### BRIEF SUMMARY OF THE INVENTION

In certain embodiments of the invention, a method of detecting a neurological event includes acquiring EEG signal data comprising a stream of data values, determining a short-term and a long-term representation of the EEG signal data, calculating a ratio of the short-term representation to the long-term representation, and comparing the ratio to a threshold. A neurological event may be detected when the ratio exceeds the threshold. The short-term representation of the EEG signal data may be determined using a multi-stage filtering process. For example, in certain embodiments, the multi-stage filtering process may include a filter that operates on successive blocks of EEG signal data values to produce intermediate output values, followed by a filter that operates on a rolling window of the intermediate output values. In certain embodiments, the long-term representation of the EEG signal data may also be determined using a multi-stage filtering process.

In an exemplary embodiment, a computer readable medium may be programmed with instructions for performing a method of detecting a neurological event, the instructions adapted to cause a programmable processor to acquire EEG signal data, determine a short-term and a long-term representation of the EEG signal data, calculate a ratio of the short-term representation to the long-term representation, and compare the ratio to a threshold to detect a neurological event when the ratio exceeds the threshold, for example.

In still another exemplary embodiment, an implantable medical device system for detecting a neurological event includes an implantable medical device (IMD) and at least one electrode adapted to communicate EEG signals to the IMD, the device being capable of acquiring EEG signal data comprising a stream of data values, determining a short-term and a long-term representation of the EEG signal data, calculating a ratio of the short-term representation to the long-term representation, and comparing the ratio to a threshold. Further embodiments may be adapted to deliver therapy to a patient when a neurological event is detected.

### BRIEF DESCRIPTION OF THE DRAWINGS

The present invention will hereinafter be described in conjunction with the following drawing figures, wherein like numerals denote like elements:

FIG. 1 shows an implantable system for treating a nervous system disorder according to an embodiment of the invention;

FIG. 2 is a schematic block diagram of an implantable medical device for treatment of a nervous system disorder in accordance with embodiments of the invention;

FIG. 3 is an exemplary EEG signal waveform, showing neurological events corresponding to epileptic seizures;

FIG. 4 shows a simulated EEG signal waveform, designating portions of a neurological event;

FIG. 5 shows an example of an EEG signal waveform and a corresponding plot of an exemplary event monitoring parameter for detecting neurological events in accordance with various embodiments of the invention;

FIG. 6 is a plot of an exemplary event monitoring parameter associated with a seizure detection algorithm according to various embodiments of the invention;

FIG. 7(a) is a block diagram of an exemplary method of determining a short-term representation of an EEG signal in accordance with certain embodiments of the invention;

FIG. 7(b) is a block diagram of an exemplary method of determining a long-term representation of an EEG signal in accordance with certain embodiments of the invention; and

FIG. 8 is a block diagram showing an exemplary method of detecting a neurological event according to an embodiment of the invention.

### DETAILED DESCRIPTION OF THE INVENTION

The following discussion is presented to enable a person skilled in the art to make and use the invention. Various modifications to the illustrated embodiments will be readily apparent to those skilled in the art, and the generic principles herein may be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the invention. Skilled artisans will recognize the examples provided herein have many useful alternatives which fall within the scope of the invention as claimed.

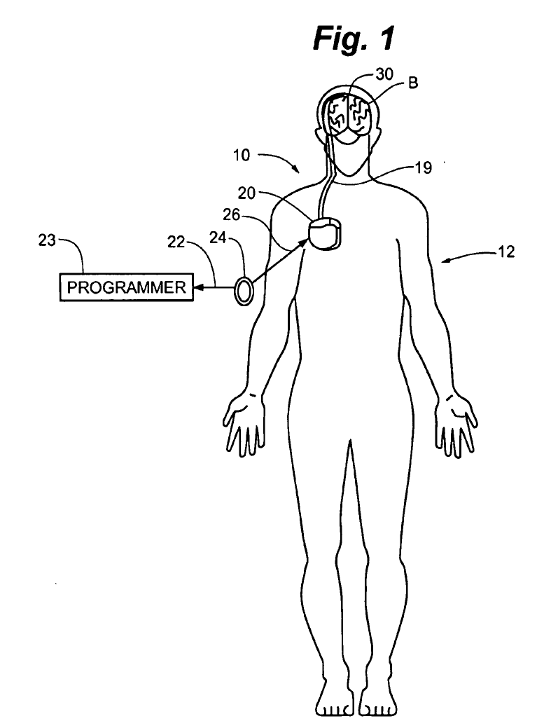

FIG. 1 shows an embodiment of an implanted system 10 for treatment of a nervous system disorder in accordance with an embodiment of the invention. System 10 includes implantable medical device (IMD) 20, lead(s) 19, and electrode(s) 30. Although the implanted system 10 is discussed herein in the context of monitoring and recording brain activity and/or providing brain stimulation, it will be appreciated that the implanted system 10 may also be used to monitor and record physiological signals from, or provide treatment therapies to, other locations of the body. The IMD 20 could, for example, be a neurostimulator device, a pacing device, a defibrillation device, an implantable loop recorder, a hemodynamic monitor, or any other implantable signal recording device known in the art or developed in the future. In FIG. 1, the IMD 20 is electrically coupled to the brain B of patient 12 through electrodes 30 and lead conductor(s) of at least one lead 19 in a manner known in the art. The electrodes 30 may also serve as therapy delivery elements to treat nervous system disorders. The IMD 20 may continuously or intermittently communicate with an external programmer 23 (e.g., patient or physician programmer, or a comparable computing and display interface) via telemetry using, for example, antenna 24 to relay radio-frequency signals 22, 26 between IMD 20 and programmer 23. In this embodiment, each of the features and functionalities discussed herein are provided by the IMD 20.

Those skilled in the art will appreciate that some medical device systems may take any number of forms from being fully implanted to being mostly external and can provide treatment therapy to any number of locations in the body, as disclosed in U.S. Pat. No. 6,341,236 (Osorio, et al.), incorporated herein by reference. For example, the medical device systems described herein may be utilized to provide treatment therapy including, for example, electrical stimulation, magnetic stimulation, and/or drug infusion. Moreover, it will be appreciated that the medical device systems may be utilized to analyze and treat any number of nervous system disorders. In the event that closed-loop feedback control is provided, the medical device system can be configured to receive any number of neurological signals that carry information about a symptom or a condition or a nervous system disorder. Such signals may be provided using one or more monitoring elements such as monitoring electrodes or sensors. For example, U.S. Pat. No. 6,227,203 provides examples of various types of sensors that may be used to detect a symptom or a condition or a nervous system disorder and responsively generate a neurological signal and is hereby incorporated by reference in relevant part.

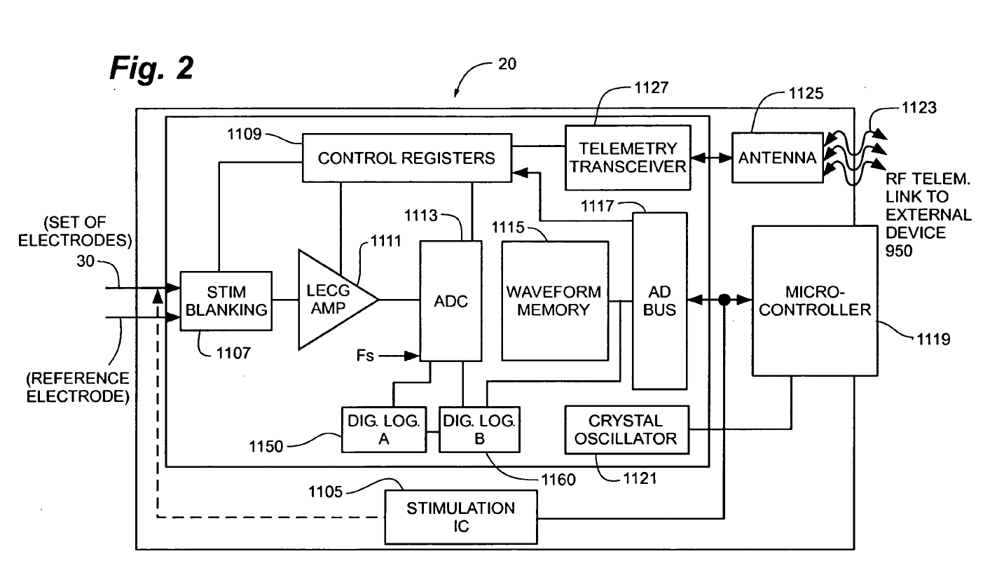

FIG. 2 is a schematic block diagram of an IMD 20. The IMD 20 is typically implanted in conjunction with a set of electrodes 30. The IMD 20 may be capable of communicating with an external device, such as programmer 23 (FIG. 1), through a telemetry transceiver 1127, an antenna 1125, and a telemetry link 1123. The external device may collect data from the IMD 20 by placing antenna 24 on the patient's body 12 over the IMD 20 to thereby communicate with IMD 20 via antenna 1125.

IMD 20 may contain an operating system that may employ a microcomputer or a digital state machine for sensing and analyzing physiological signals in accordance with a programmed operating mode. The IMD 20 may also contain sense amplifiers for detecting signals, and output circuits for delivering electrical stimulation therapy, for example, to certain parts of the brain B. The operating system may include a storage device for storing sensed physiological signals, including those associated with neurological activity. The storage device may also be used for storing operating parameters and other operating history data.

Each electrode of the set of electrodes 30 may be adapted to either receive a physiological signal, such as a neurological signal, or to stimulate surrounding tissue, or to perform both functions. Stimulation of any of the electrodes contained in the electrode set 30 is generated by a stimulation IC 1105, as instructed by a microcontroller (or microprocessor) 1119. When stimulation is generated through an electrode, the electrode may be blanked by a blanking circuit 1107 so that a physiological signal is not received by channel electronics (e.g., amplifier 1111). U.S. Patent Application Publication 2004/0133248 to Frei et al. (“Channel-Selective Blanking for a Medical Device System”), incorporated by reference herein, discloses a method of blanking signal channels during the delivery of therapy. When microprocessor 1119 determines that a channel is able to receive a physiological signal, an analog to digital converter (ADC) 1113 samples the physiological signal at a desired rate (e.g., about 200 to 250 samples per second per channel, according to some embodiments). Digital logic circuitry, indicated in FIG. 2 by digital logic 1150 and 1160, may be employed to receive the digitized physiological signal from ADC 1113. The digitized physiological signal may be stored in a waveform memory 1115 so that the neurological data may be retrieved from the IMD 20 when instructed, or may be processed by microprocessor 1119 to generate any required stimulation signal. In some embodiments, digital logic 1150, 1160 may employ a data compression step, such as applying the new turning point (NTP) algorithm or other suitable data compression algorithms or filters, to thereby reduce memory constraints that may be imposed on an IMD due to issues of size, power consumption, and cost, for example.

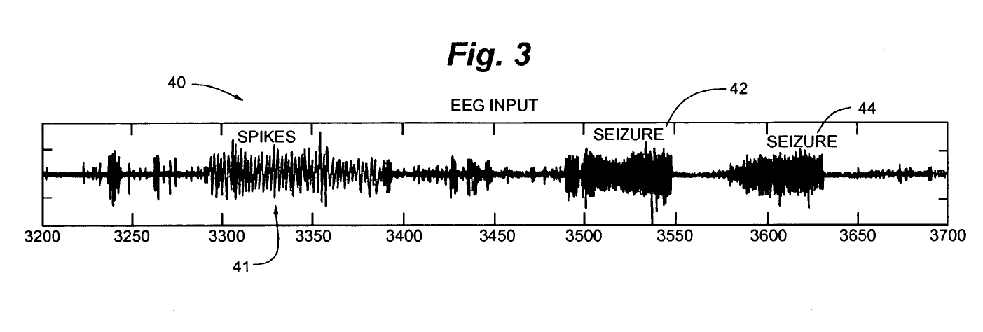

FIG. 3 shows an example of an EEG signal waveform 40. Epileptic seizures 42, 44 may manifest as changes in EEG signal amplitude energy and/or frequency from an underlying EEG rhythm, as shown in FIG. 3. Also shown are epileptiform discharge spikes 41, which may occur prior to the occurrence of seizures 42, 44. In certain cases, neurological events, such as seizures 42 and 44, may be thought of as belonging to a single group or cluster of events, for example. Associating a group of events as belonging to a single cluster may, for example, be useful in making decisions regarding therapy delivery.

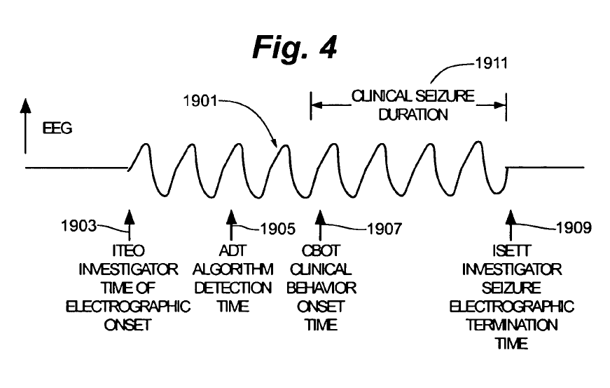

FIG. 4 shows a simulated EEG waveform 1901, designating portions of an exemplary neurological event. A time event 1903 corresponds to an investigator time of electrographic onset (ITEO), indicating a point at which a clinician may observe a significant amount of electrographic activity on an EEG waveform 1901 that may mark the beginning of a neurological event such as a seizure. (However, a neurological event may not necessarily follow time event 1903 in some cases.) A time event 1905 corresponds to an algorithm detection time (ADT), indicating a point at which a detection algorithm may detect an occurrence of a neurological event based on processing of an EEG waveform 1901.

A time event 1907, illustrated in FIG. 4, corresponds to a clinical behavior onset time (CBOT), indicating a point at which a patient typically manifests the symptoms of a neurological event (such as demonstrating the physical characteristics of a seizure). However, in some cases, a patient may not manifest symptoms even though an ITEO occurs. Typically, if monitoring elements (such as electrodes) are appropriately positioned, the CBOT 1907 will occur after the ITEO 1903. However, depending on the placement of the electrodes relative to the location of the neurological event, the CBOT 1907 may occur before the ITEO 1903 due to potential delays of neurological signals propagating through various portions of a patient's brain. With continued reference to FIG. 4, a time event 1909 corresponds to an investigator seizure electrographic termination time (ISETT), in which the electrographic activity decreases to a level low enough to indicate termination of seizure activity. A time interval 1911 is also depicted in FIG. 4 to indicate clinical seizure duration, which may be defined as the time interval from CBOT 1907 to ISETT 1909.

Overview of IMD System

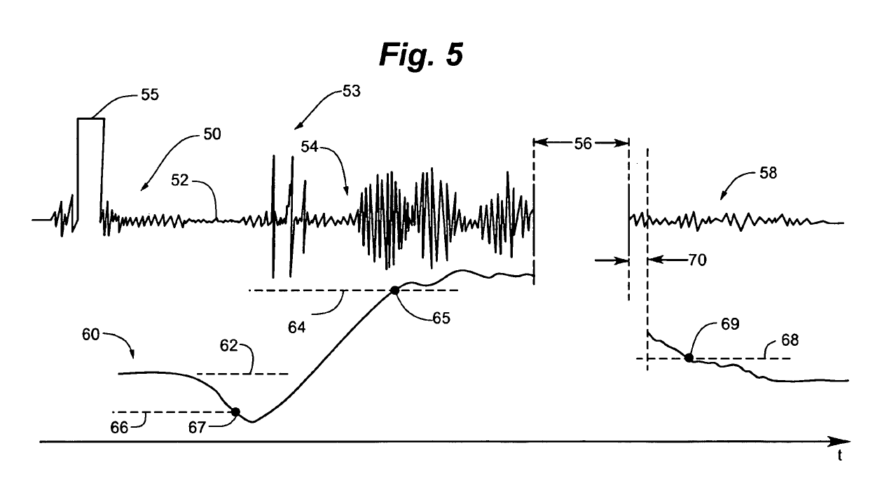

FIG. 5 shows a time plot that generally illustrates the operation of an IMD system in response to an EEG signal in accordance with certain embodiments of the invention. A single channel EEG signal 50 is shown in the top plot spanning a period of time that includes pre-seizure activity, seizure onset, therapy delivery, and post-therapy monitoring of EEG signal 50. The bottom plot is an exemplary event monitoring parameter 60 that may be derived from one or more channels of EEG signals 50. The event monitoring parameter may also be referred to as a seizure monitoring parameter. FIG. 5 shows event monitoring parameter 60 starting from a relatively stable or normal value 62, corresponding to normal EEG signal activity or signal energies (e.g., during interictal periods). As shown, parameter 60 may increase or decrease due to changes in signal energy, and may cross one or more predefined threshold values 64, 66 to indicate the onset (or potential onset) of an epileptic seizure. Parameter 60 is shown crossing threshold 64 at point 65 to indicate the onset of an epileptic seizure 54, in this case identified by an increase in parameter 60 above a seizure onset threshold 64. In some embodiments, a seizure detection algorithm may also require the parameter 60 to exceed the threshold 64 for a specified duration (not shown) in order for the IMD to “detect” the seizure.

Similarly, parameter 60 is also shown dropping below threshold 66 at point 67 in FIG. 5 to indicate the possible onset of a seizure according to certain embodiments of the invention. A specified duration parameter may also be required to be met in order to detect a seizure based on this type of threshold criterion. As shown, a low-level threshold such as threshold 66 may be used to indicate a low level of EEG signal energy, as shown at 52, which may be used as an early predictor or precursor of an epileptic seizure in some embodiments of the invention.

The EEG signal 50 in FIG. 5 also shows epileptiform discharge spikes 53, which may also serve as an early predictor or precursor of an epileptic seizure. Certain embodiments of the invention include a method (not shown in FIG. 5) for analyzing the occurrence of such spikes 53 and using them to “detect” a precursor to (e.g., to “predict”) a possible seizure.

The methods of detecting neurological events (such as seizures and seizure precursors) described herein may be affected by the quality of the signals employed by the various methods. For example, periods of signal saturation or clipping, as indicated in EEG signal 50 at point 55, may provide false information to a seizure detection algorithm. Systems and methods for monitoring and accounting for signal quality are disclosed in U.S. Patent Application Publications 2004/0138580 and 2004/0138581 to Frei et al. (both entitled “Signal Quality Monitoring and Control for a Medical Device System”), both of which are hereby incorporated by reference in their respective entireties.

FIG. 5 also illustrates the delivery of therapy 56 from an IMD system in response to a detected seizure. The IMD system may provide therapy in the form of electrical stimulation to portions of the nervous system, or in the form of drug delivery, or in other forms of therapy suitable for the treatment of an epileptic seizure. FIG. 5 further illustrates the resumption of EEG signal monitoring following the delivery of therapy 56 to a patient, as shown in the EEG signal at 58. After successful therapy delivery by the IMD system (or after the neurological event terminates on its own), parameter 60 may drop below a seizure termination threshold 68 as shown at point 69 to indicate the end of the seizure. In some embodiments, the IMD system may require that parameter 60 remain below seizure termination threshold 68 for a predetermined period of time to mark the end of a seizure, according to certain embodiments of the invention (e.g., a predefined duration).

An additional or optional aspect of an IMD in accordance with various embodiments of the invention is also indicated by post-stimulation interval 70 in FIG. 5. For example, at the termination of therapy 56, the IMD may not immediately have data available from which to derive or calculate parameter 60 (or data may be “old” data received prior to stimulation therapy, for example). In some embodiments, this may be at least temporarily addressed by an alternate means of determining parameter 60 (or a substitute parameter) after the delivery of therapy 56, which may quickly assess whether a seizure is still on-going and/or determine the need for additional stimulation therapy, for example.

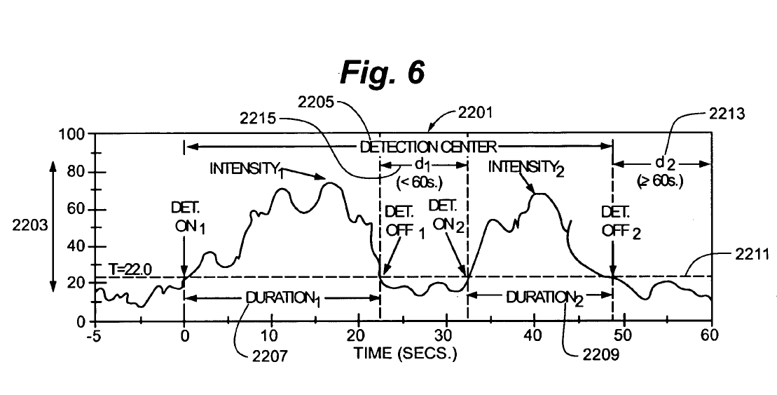

FIG. 6 shows a pair of neurological events detected using a method in accordance with certain embodiments of the invention. During a neurological event (such as a seizure), EEG activity, as monitored with a seizure detection algorithm, may result in multiple closely-spaced detections or clusters that a physician/clinician may wish to interpret as being related as part of a single event (e.g., one episode), and which, if considered as separate events, may result in an unnecessary therapy delivery, or possibly an unsafe number of therapy deliveries. This may be particularly true at the beginning or end of a neurological event when oscillations around the detection threshold may result in multiple closely-spaced detections, which may complicate operations and logging of events.

A medical device system, e.g., IMD 20, may associate clusters of closely-spaced detections using a temporal criterion. For example, detections that are separated in time by less than a programmable inter-detection interval may be classified as being related, and/or may be deemed to be part of the same cluster or episode. Parameters, such as an inter-detection interval, may be programmable in IMD 20, for example. U.S. Patent Application Publication 2004/0138536 to Frei et al. (“Clustering of Neurological Activity to Determine Length of a Neurological Event”), hereby incorporated by reference in its entirety, discloses such a method of detecting a cluster or clusters of neurological events.

FIG. 6 shows data 2201 associated with an event monitoring parameter 2203, which may be determined by a seizure detection algorithm. A pair of detections is shown, including two periods (Duration₁, at 2207, and Duration₂, at 2209) during which event monitoring parameter 2203 exceeds a threshold 2211, as well as a relatively brief intervening period, d₁, between 2207 and 2209. Event monitoring parameter 2203 is displayed in FIG. 6 from about 5 seconds before the onset of the first detection to about 12 seconds after the end of the second detection. A number of methods of determining event monitoring parameter 2203 from one or more EEG signals are described below in later sections.

In certain embodiments, a time constraint may be defined such that, if event monitoring parameter 2203 falls below predetermined threshold 2211 (e.g., after a first detected event), then subsequently rises above predetermined threshold 2211 (e.g., a second detection occurs) within the defined time constraint, then that subsequent detection is considered to be related to the first detection (e.g., part of the same detection cluster). Thus, the pair of detections 2205 includes Duration₁ 2207, the intervening interval, d₁, and Duration₂ 2209. Analysis of the event monitoring parameter 2203 (and therapy decision based thereon) may therefore be performed on clusters or groups of detections, rather than solely on individual detected events.

Seizure severity metrics (e.g., measures of the intensity of a detected seizure) may be determined based on analysis of the event monitoring parameter 2203 over an entire cluster 2205 (rather than on individual detected events). For example, a severity metric may be defined as the maximum value of event monitoring parameter 2203 reached during cluster 2205 in certain embodiments. U.S. Patent Application Publication 2004/0133119 to Osorio et al. (“Scoring of Sensed Neurological Signals for use with a Medical Device System”), hereby incorporated by reference in its entirety, discloses such a method of scoring the severity of sensed neurological signals.

Referring again to FIG. 2, ADC circuit 1113 receives the physiological signal from electrodes 30, which, in certain embodiments, may be sampled at appropriate rates, such as about 256 or 128 Hz or samples per second. Sampling physiological signals at rates above about 128 Hz is usually adequate to avoid “aliasing” because there is typically little signal energy above 60 Hz included in the sampled signal. “Aliasing” is a phenomenon of the digitization process that may be caused by sampling at too low a sample rate for a given signal, resulting in reproduced signals with spurious or erroneous frequency content. Aliasing is typically avoided by performing analog low-pass filtering prior to digitization to limit the frequency content, then sampling at a rate greater than about twice the frequency of the highest frequency content in the filtered signal. For example, the upper frequency corner of the analog filter should be no more than half of the sample rate (by Nyquist's Law), and is usually lower than that.

Data signals stored by the IMD 20 may be transmitted between an IMD RF telemetry antenna 1125 (FIG. 2) and an external RF telemetry antenna 24 associated with the external programmer 23 (FIG. 1). In an uplink telemetry transmission 22, the external RF telemetry antenna 24 operates as a telemetry receiver antenna, and the IMD RF telemetry antenna 1125 operates as a telemetry transmitter antenna. Conversely, in a downlink telemetry transmission 26, the external RF telemetry antenna 24 operates as a telemetry transmitter antenna, and the IMD RF telemetry antenna 1125 operates as a telemetry receiver antenna. Both RF telemetry antennas 24 and 1125 are coupled to a transceiver including a transmitter and a receiver. This is as described in commonly-assigned U.S. Pat. No. 4,556,063, herein incorporated by reference in relevant part.

Implantable Seizure Detection Algorithm

As noted above with respect to FIG. 5, an event monitoring parameter 60 may be derived from one or more EEG signals to form the basis of a seizure detection algorithm. Several event monitoring parameters 60 may be derived and used concurrently in certain embodiments, for example, by combining several such parameters using logical functions (e.g., AND, OR, MAX, MIN, etc.). In the sections that follow, a ratio method and an evidence counter method are described, either or both of which may be used by an IMD to detect the onset of neurological events such as seizures. Several methods are also described below which may anticipate or predict neurological events, for example, by detecting one or more precursors of seizure activity.

Seizure Detection—Ratio Method

Adverse neurological events, such as epileptic seizures, are typically characterized by increases in EEG signal energy (including increases in signal amplitude and/or frequency). An increase in EEG signal energy (e.g., within a specified frequency range) may be identified or detected, for example, relative to a reference or background level of EEG signal energy. An event monitoring parameter may therefore be defined as a ratio of a relatively recent, short-term representation of an EEG signal (e.g., the “foreground” or “FG”) to a relatively long-term representation of an EEG signal (e.g., the “background” or “BG”). The short-term and long-term representations may be indicative of EEG signal amplitude, energy, and/or frequency, according to various embodiments of the invention.

The foreground may, for example, be determined from analysis of an EEG signal acquired over a first sample interval. The first sample interval may be a relatively recent, relatively brief time window in certain embodiments of the invention. In one particular embodiment, a recent two-second time window may be used as the first sample interval for calculating the foreground. In certain embodiments, a median value of the EEG signal magnitude over the two-second window may be used as the foreground. Of course, shorter or longer time windows can be chosen from which to base the determination of the foreground, as would be apparent to one of ordinary skill in the art. Similarly, statistical measures other than the median (e.g., mean, root-mean-square, weighted average, rank order or Xth percentile, etc.) may be used to determine a value for the foreground.

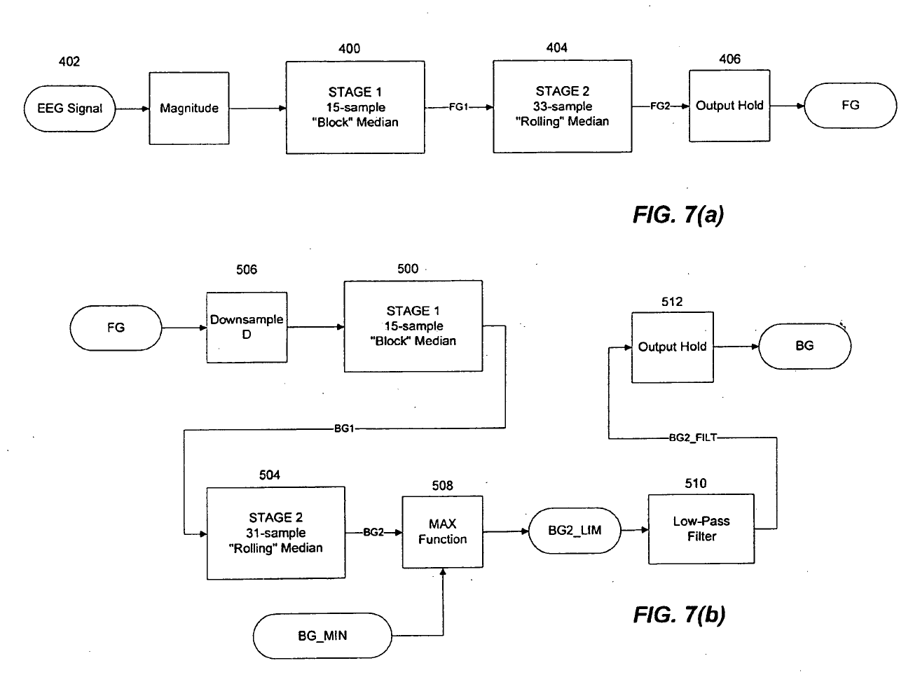

In certain embodiments of the invention, a method of determining a median (or other suitable statistical measure) may employ a multi-stage or cascading technique in order to simplify the calculations, to thereby conserve computational resources (e.g., memory, processor speed/capabilities, and battery capacity) which may be limited in many IMDs due to constraints on device size; reductions in the size of an IMD may also be made possible by efficient use of computing resources. One such cascading technique that may be used in accordance with certain embodiments of the invention includes a two-stage filter, as described in the flow diagram of FIG. 7(a). In the embodiment shown, the first stage filter 400 receives as an input an EEG signal 402, comprising a stream of data values. The stream of data samples may be obtained from a narrowband filtered digital EEG signal, for example. The first filter 400 processes “blocks” of incoming EEG signal data values and determines a statistical measure, such as the median value, of each block, and produces this as an intermediate output value. A block may correspond to a specified number, N, of EEG signal data values. The example shown in FIG. 7(a) uses a block of 15 incoming EEG signal data values, obtained by digitally sampling an EEG signal at a sample rate of approximately 250 samples per second. Other values could be used for the block size, N, and/or the sample rate, as would be apparent to those of ordinary skill in the art. In some embodiments, a block may have a value of N from 3 to 25 data values, and may preferably have from 11 to 21 data values, and more preferably, about 15 data values.

The intermediate output value of the first filter 400, FG1, is then input to a second stage filter 404. The second filter 404 may be a “rolling” filter which computes a median (or other suitable statistical measure) based on a rolling window containing a specified number, M, of the intermediate output values (e.g., the FG1 values). In the example shown, the rolling window comprises 33 FG1 intermediate output values (e.g., the rolling window may be a FIFO buffer of length 33), corresponding to a time window (or first sample interval) of approximately two seconds. The output of the second filter 404, FG2, will therefore update with each new FG1 intermediate output value provided as an input (e.g., roughly every 15/250 seconds in this example). In some embodiments, the number of intermediate output values, M, in a rolling window may range from about 3 to 101 intermediate output values, and may preferably range from about 11 to 51 intermediate output values, and more preferably, range from about 29 to 35 intermediate output values.

In an optional embodiment, an output hold function 406 may be applied to the FG2 short-term representation values to hold the value between updates, and to thereby produce the foreground signal, FG, as shown in FIG. 7(a). In the example described, a first stage memory buffer of length 15 and a second stage buffer of length 33 are used, rather than a single buffer of length 495, to determine the median value of the incoming signal samples over the foreground interval. This may result in more efficient usage of computing resources. Of course, one of ordinary skill in the art would recognize that more than two stages of filters may be used, as well as memory buffers of different lengths than those described above, to determine a value that is a short-term representation of the EEG signal for use in detecting a neurological event. Further, a statistical measure other than the median may be employed to determine representations of the EEG signal, such as the mean, minimum, maximum, and other suitable statistical measures, for example.

A relatively long-term representation of the EEG signal (e.g., the background) may also be calculated, for example, using a two-stage filter similar to that described above with respect to the foreground determination. The background may be derived from EEG signal data values accumulated over a second sample interval spanning a relatively long period of time (i.e., longer than the first sample interval). For example, a 20-minute or 30-minute period may be appropriate for the second sample interval according to some embodiments. Of course, longer or shorter periods may also be used. A stream of input values derived from the EEG signal data values may then be applied to a two-stage filter to determine the background signal, BG. In certain embodiments, the foreground signal, FG (see FIG. 7(a)), may be used to form the stream of input values derived from the EEG signal data values for determining the background signal, BG. The two-stage filter may form intermediate background values, BG1 and BG2, as shown in FIG. 7(b). FIG. 7(b) shows a filter 500 as a block filter, which may be adapted to produce BG1 intermediate output values based on blocks of a specified number, X, of input values (15 successive input values in the example shown). For example, each BG1 intermediate output value produced by filter 500 may be the median (or other suitable statistical measure) of the X input values in a given block. The specified number of input values, X, in a block may be any desired value. In some embodiments, a block may have from 3 to 25 data values, and may preferably have from 11 to 21 data values, and more preferably, about 15 data values.

A filter 504 is next shown as a rolling window filter which receives BG1 intermediate output values and produces BG2 values based on rolling windows having a specified number, Y, of BG1 intermediate output values (31 intermediate output values in the example shown). Of course, the number of samples, blocks, and stages may be varied by one of ordinary skill in the art without departing from the scope of the invention as claimed.

In some embodiments, an optional step of downsampling 506 of the FG values may be performed prior to applying them to the filter 500 to reduce the computational complexity and/or improve efficiency. In the example shown, a downsampling factor, D, may be used to select every D^{th} sample from FG as an input to the filter 500. A downsampling factor of 2 in the example above may result in every other FG value being used as an input to filter 500 such that the 15-sample block median filter output, BG1, corresponds to approximately one minute of data, and the 31-sample rolling median output, BG2, corresponds to roughly 30 minutes of data. Of course, the specific numerical values used in the above examples are merely exemplary, and are provided for purposes of illustration, not limitation.

FIG. 7(b) shows an optional embodiment in which the second stage output, BG2, is compared to a specified minimum value, BG_MIN, using a maximum function 508 to produce an output, BG2_LIM, that is the greater of BG2 and BG_MIN. This step, if employed, effectively places a floor on the value of BG2_LIM so that it cannot drop below the value BG_MIN. A further optional step may include passing BG2_LIM through a low-pass filter 510 to produce a filtered background output, BG2_FILT. Next, the background signal, BG2_FILT (or BG2, or BG2_LIM, depending on the particular embodiment), may be applied to a hold function 512 to maintain the output value between successive updates, thereby producing the background signal, BG.

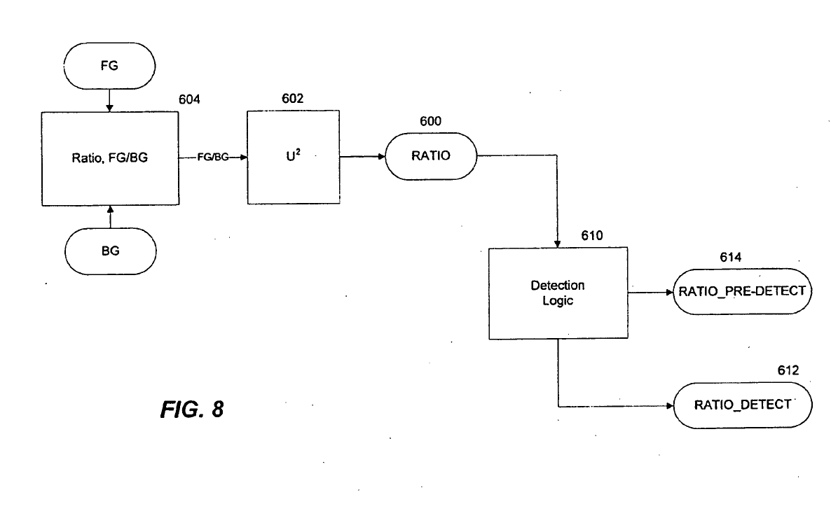

As mentioned above, a ratio of foreground and background signal energies may be defined and used as a criterion for detecting neurological events, such as epileptic seizures. FIG. 8 illustrates one possible embodiment of the invention in which a ratio 600 is computed from the above-described foreground and background signals, FG and BG. In the embodiment shown, the ratio may be determined by dividing the foreground FG by the background BG at function 604, then optionally squaring the result as shown by the squaring function, U² 602 to produce ratio 600. In certain embodiments, the foreground and background signals, FG and BG, may each be squared (by a function similar to function 602) prior to forming the ratio 600. As would be apparent to one of ordinary skill in the art, other similar functions may be used to determine ratio 600. For example, the optional squaring function 602 may be omitted and/or replaced with other functions, such as an absolute value function, or a difference function, or a squared difference function, or combinations of these and other functions.

In certain embodiments of the invention, determining the value of ratio 600 may be performed by a method that estimates the ratio using an exponential approximation technique substantially as described in commonly assigned U.S. patent application Ser. No. 10/976,474. According to this technique, a ratio of a numerator (e.g., the short-term representation or FG) to a denominator (e.g., the long-term representation or BG) may be estimated by raising the number 2 to an exponent value, the exponent value being equal to the difference in the most significant set bit (MSSB) positions of the denominator and numerator, respectively. The MSSB position may be defined as the numbered bit position of a first non-zero bit in a binary number, starting from the most significant bit (MSB) of that number. For example, the exponent value may be obtained by determining the difference between the MSSB position of the long-term representation and the MSSB position of the short-term representation. The following example illustrates the use of this technique.

Numerator: 01000011 (equals 67 in decimal notation)

Denominator: 00010001 (equals 17 in decimal notation)

The MSSB of the numerator is 2, since the second bit position holds the first non-zero bit, starting from the MSB (i.e., the left-most bit). The MSSB of the denominator is 4, since the fourth bit position holds the first non-zero bit, starting from the MSB. Applying the technique, an estimate of the ratio of the numerator to the denominator is obtained by raising the number 2 to an exponent value equal to MSSB_{denominator}−MSSBₙᵤₘₑᵣₐₜₒᵣ=4−2=2. Thus, the estimate is 2²=4, which is reasonably close to the value of 67/17 in this example. Of course, various refinements and minor modifications to the technique described may be employed by one of ordinary skill in the art to determine a ratio value in accordance with embodiments of the invention, and would be considered to fall within the scope of the invention as claimed.

The onset of a neurological event (e.g., a seizure) may be detected when a predefined ratio 600 of foreground and background signal levels (or a function derived therefrom) crosses or exceeds an onset threshold. In certain embodiments, detection of a seizure may further require that the ratio 600 exceed the onset threshold for a specified period of time (e.g., duration), according to certain embodiments of the invention. This is shown as detection logic 610 in FIG. 8. Seizure detection logic 610 may further include a seizure termination threshold and optionally a seizure termination duration parameter which may be used to indicate the end of a seizure episode, for example, when the ratio 600 falls below the termination threshold for a period longer than the termination duration. The threshold and duration parameters may be pre-defined and/or user-selectable, and need not be the same for onset and termination.

FIG. 8 also shows the output of detection logic 610 as including two possible outputs, RATIO_DETECT 612 and RATIO_PRE_DETECT 614. RATIO_PRE_DETECT 614 may change from a logical value of “False” (e.g., a value of 0) to a logical value of “True” (e.g., a value of 1) when the ratio 600 first exceeds the onset threshold, for example. This may be useful, for example, to trigger an event such as charging up electrical stimulation circuitry in preparation for therapy delivery. If the ratio 600 exceeds the onset threshold for the onset duration, the RATIO_DETECT 612 value may also change from a logical value of “False” (e.g., a value of 0) to a logical value of “True” (e.g., a value of 1). In some embodiments, therapy may be delivered when the ratio has exceeded the onset threshold for the specified duration. RATIO_DETECT 612 and RATIO_PRE_DETECT 614 may both return to “False” (e.g., a value of 0) if the ratio 600 falls below a predetermined termination threshold. Some embodiments may also require that the ratio 600 remain below the termination threshold for a predetermined duration before assigning a logical value of “False” to the RATIO_DETECT 612 and RATIO_PRE_DETECT 614.

In embodiments using a duration parameter, either for the onset threshold or the termination threshold, duration may be defined in a number of ways. For example, to satisfy the duration parameter, the method may require that a specified number of consecutive ratio 600 values exceed the threshold value before the duration is satisfied. Alternately, the duration parameter may be defined to require that consecutive ratio values meet the respective threshold criteria for a specified period of time. In other embodiments, the duration parameter may be defined such that duration is satisfied, for example, by having at least a certain number of ratio values within a predefined window of ratio values that exceed the respective threshold values (e.g., a predetermined percentage of values of the ratio must exceed the threshold for the given duration parameter). For example, a duration criterion may require that seven out of a rolling window of ten ratio values exceed the respective threshold value in order to satisfy the duration criterion. Other possibilities exist for devising a duration criterion, as would be apparent to one of ordinary skill in the art with the benefit of these teachings.

The use of a ratio parameter 600 as a detection criterion may typically detect seizures a few seconds after the electrographic onset. It is hypothesized that therapy effectiveness may diminish the longer therapy is delayed from onset. Therefore, to minimize the delay between detection of a seizure and delivery of therapy (e.g., electrical stimulation), the output stimulus circuits in an IMD may be adapted to begin charging prior to seizure detection. For example, the output stimulus circuits may receive instructions to begin charging when RATIO_PRE_DETECT 614 becomes True (e.g., a logical value of 1) in embodiments where this marks the beginning of a duration criteria. Thus, the output stimulus circuits may have time to become at least partially charged prior to satisfying a seizure onset duration parameter, according to some embodiments of the invention. This may, for example, allow enough time for the stimulus circuits to become fully charged and ready to deliver stimulation therapy immediately after duration is satisfied and/or RATIO_DETECT 612 becomes “True.”

The ratio parameter 600 may also be used to determine whether a group of detected neurological events are related, for example, as part of a single seizure cluster or episode. For example, a given neurological event may be considered to be part of the same seizure cluster or episode as the immediately preceding neurological event if the amount of time that elapses from the end of the immediately preceding neurological event to the given neurological event is less than a predefined cluster timeout interval, as discussed above with respect to FIG. 6.

Thus, a METHOD AND APPARATUS FOR DETECTION OF EPILEPTIC SEIZURES has been provided. While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method of detecting a neurological event, comprising:
acquiring EEG signal data comprising a stream of data values;
determining a short-term representation of the EEG signal data based on data values acquired over a first sample interval, the short-term representation being determined by applying the data values to a first filter, the first filter adapted to produce an intermediate output value for each block of N data values, and applying a rolling window of M intermediate output values to a second filter, the second filter adapted to produce the short-term representation;
determining a long-term representation of the EEG signal data based on data values acquired over a second sample interval, the second sample interval being longer than the first sample interval;
calculating a ratio of the short-term representation to the long-term representation;
comparing the ratio to an onset threshold; and
detecting a neurological event when the ratio exceeds the onset threshold.

2. The method of claim 1 wherein the neurological event is an epileptic seizure.

3. The method of claim 1 wherein the long-term representation is determined by applying a stream of input values derived from the data values to a third filter, the third filter adapted to produce an intermediate output value for each block of X input values, and applying a rolling window of Y intermediate output values to a fourth filter, the fourth filter adapted to produce the long-term representation.

4. The method of claim 3 wherein the stream of input values comprise the short-term representation values.

5. The method of claim 4 wherein the short-term representation values are downsampled prior to being applied to the third filter.

6. The method of claim 5 wherein the short-term representation values are downsampled by a downsampling factor of 2.

7. The method of claim 3 further comprising setting the value of the long-term representation to a specified minimum value if the value determined using the third and fourth filters is less than the specified minimum value.

8. The method of claim 7 further comprising applying a low-pass filter to the value of the long-term representation.

9. The method of claim 1 wherein determining the ratio of the short-term representation to the long-term representation comprises estimating the ratio as a function of 2 raised to an exponent, the exponent comprising a difference between a most significant set bit (MSSB) position of the long-term representation and a MSSB position of the short-term representation, wherein the MSSB position is a numbered bit position of a first non-zero bit, starting from a most significant bit (MSB).

10. The method of claim 1 wherein the intermediate output value produced by the first filter is a statistical measure of the data values in a given block.

11. The method of claim 10 wherein the statistical measure is a median value.

12. The method of claim 10 wherein the statistical measure is a mean value.

13. The method of claim 1 wherein the short-term representation produced by the second filter is a statistical measure of the data values in the rolling window.

14. The method of claim 13 wherein the statistical measure is a median value.

15. The method of claim 13 wherein the statistical measure is a mean value.

16. The method of claim 1 wherein the first sample interval for determining the short-term representation is a period of less than about 5 minutes.

17. The method of claim 16 wherein the first sample interval for determining the short-term representation is a period of about 2 minutes.

18. The method of claim 1 wherein the second sample interval for determining the long-term representation is a period of greater than about 15 minutes.

19. The method of claim 18 wherein the second sample interval for determining the long-term representation is a period of greater than about 20 minutes.

20. The method of claim 18 wherein the second sample interval for determining the long-term representation is a period of about 30 minutes.

21. The method of claim 3 wherein the third and fourth filters used to determine the long-term representation produce statistical measures of the data values applied to each filter.

22. The method of claim 1 wherein a neurological event is detected when the ratio exceeds the onset threshold for an onset duration.

23. The method of claim 22 wherein a neurological event is detected when a specified number of consecutive values of the ratio exceeds the onset threshold for an onset duration.

24. The method of claim 22 wherein a neurological event is detected when a predetermined percentage of values of the ratio exceeds the onset threshold for a detection duration.

25. The method of claim 1 further comprising identifying an end of a neurological event when the ratio decreases below a termination threshold.

26. The method of claim 25 wherein the end of a neurological event is identified when the ratio decreases below a termination threshold for a termination duration.

27. The method of claim 26 wherein the end of a neurological event is identified when a specified number of consecutive values of the ratio decreases below the termination threshold for a termination duration.

28. The method of claim 26 wherein the end of a neurological event is identified when a predetermined percentage of values of the ratio decreases below the termination threshold for a termination duration.

29. The method of claim 1 wherein the onset threshold is greater than 5.

30. The method of claim 29 wherein the onset threshold is between about 10 and 50.

31. The method of claim 30 wherein the onset threshold is about 22.

32. The method of claim 1 wherein the number of data values N in each block is a specified number in a range from 3 to 25 data values.

33. The method of claim 32 wherein the number of data values N in each block is a specified number in a range from 11 to 21 data values.

34. The method of claim 33 wherein the N data values in each block includes 15 successive data values.

35. The method of claim 1 wherein the number of intermediate output values M in a rolling window is a specified number in a range from 3 to 101 intermediate output values.

36. The method of claim 35 wherein the number of intermediate output values M in a rolling window is a specified number in a range from 11 to 51 intermediate output values.

37. The method of claim 36 wherein the intermediate output values M in a rolling window include 29 to 35 successive intermediate output values.

38. The method of claim 35 wherein the M intermediate output values are representative of a period of less than about 5 minutes.

39. The method of claim 38 wherein the M intermediate output values are representative of a period of about 2 minutes.

40. The method of claim 3 wherein the number of data values X in each block is a specified number in a range from 3 to 25 data values.

41. The method of claim 40 wherein the number of data values X in each block is a specified number in a range from 11 to 21 data values.

42. The method of claim 41 wherein the X data values in each block includes 15 successive data values.

43. The method of claim 3 wherein the number of intermediate output values Y in a rolling window is a specified number in a range from 3 to 101 intermediate output values.

44. The method of claim 43 wherein the number of intermediate output values Y in a rolling window is a specified number in a range from 11 to 51 intermediate output values.

45. The method of claim 44 wherein the intermediate output values Y in a rolling window include 29 to 35 successive intermediate output values.

46. The method of claim 43 wherein the Y intermediate output values are representative of a period of greater than about 15 minutes.

47. The method of claim 46 wherein the Y intermediate output values are representative of a period of greater than about 20 minutes.

48. The method of claim 47 wherein the Y intermediate output values are representative of a period of about 30 minutes.

49. A computer-readable medium programmed with instructions for performing a method of detecting a neurological event, the medium comprising instructions for causing a programmable processor to:
acquire EEG signal data comprising a stream of data values;
determine a short-term representation of the EEG signal data based on data values acquired over a first sample interval, the short-term representation being determined by applying the data values to a first filter, the first filter adapted to produce an intermediate output value for each block of N data values, and applying a rolling window of M intermediate output values to a second filter, the second filter adapted to produce the short-term representation;
determine a long-term representation of the EEG signal data based on data values acquired over a second sample interval, the second sample interval being longer than the first sample interval;
calculate a ratio of the short-term representation to the long-term representation;
compare the ratio to an onset threshold; and
detect a neurological event when the ratio exceeds the onset threshold.

50. The medium of claim 49 further comprising instructions to determine the long-term representation by applying the short-term representation values to a third filter, the third filter adapted to produce an intermediate output value for each block of X input values, and applying a rolling window of Y intermediate output values to a fourth filter, the fourth filter adapted to produce the long-term representation.

51. The medium of claim 49 further comprising instructions to determine the ratio of the short-term representation to the long-term representation by estimating the ratio as a function of 2 raised to an exponent, the exponent comprising a difference between a most significant set bit (MSSB) position of the long-term representation and a MSSB position of the short-term representation, wherein the MSSB position is a numbered bit position of a first non-zero bit, starting from a most significant bit (MSB).

52. An implantable medical device system for detecting a neurological event, the system comprising:
an implantable medical device (IMD); and
at least one electrode adapted to sense EEG signals from a brain of a patient and communicate the EEG signals to the device,
wherein the device is adapted to
acquire EEG signal data comprising a stream of data values;
determine a short-term representation of the EEG signal data based on data values acquired over a first sample interval, the short-term representation being determined by applying the data values to a first filter, the first filter adapted to produce an intermediate output value for each block of N data values, and applying a rolling window of M intermediate output values to a second filter, the second filter adapted to produce the short-term representation;
determine a long-term representation of the EEG signal data based on data values acquired over a second sample interval, the second sample interval being longer than the first sample interval;
calculate a ratio of the short-term representation to the long-term representation;
compare the ratio to an onset threshold; and
detect a neurological event when the ratio exceeds the onset threshold.

53. The system of claim 52 comprising at least two electrodes adapted to sense at least two channels of EEG signals, wherein the IMD is adapted to calculate the ratio of the short-term representation to the long-term representation for the at least two channels, and compare the at least two ratios to an onset threshold.

54. The system of claim 53 wherein the IMD is adapted to detect a neurological event when the ratio of at least one channel exceeds the onset threshold.

55. The system of claim 53 wherein the IMD is adapted to detect a neurological event when a maximum ratio value exceeds the onset threshold, the maximum ratio value equal to a maximum of the ratio values.

56. The system of claim 52 wherein the IMD is further adapted to deliver therapy after a neurological event is detected.

57. The system of claim 56 wherein the IMD is adapted to deliver drug therapy.

58. The system of claim 56 wherein the IMD is adapted to deliver electrical stimulation therapy.

59. The system of claim 58 wherein the IMD is adapted to deliver electrical stimulation therapy via at least one electrode also used for sensing EEG signals.

60. The system of claim 58 wherein the IMD is adapted to begin charging electronic circuitry when a neurological event is detected.
